(19) United States
(12) Patent Application Publication
(10) Pub. No.: US 2005/0112724 A1
(43) Pub. Date: May 26, 2005

(54) Pseudopterosin production

(76) Inventor: Russell Kerr, Boca Raton, FL (US)

Correspondence Address: AKERMAN SENTERFITT, P.O. BOX 3188, WEST PALM BEACH, FL, 33402-3188, US

(21) Appl. No.: 10/875,262

(22) Filed: Jun. 24, 2004

Related U.S. Application Data
(60) Provisional application No. 60/482,095, filed on Jun. 24, 2003.

(51) Int. Cl. C12N 1/12

(52) U.S. Cl. 435/69.1; 435/257.1

(57) ABSTRACT

A method for producing high levels of pseudopterosins without the need for harvesting coral from which pseudopterosins are traditionally obtained. Compositions comprising cultured alga cells produce high levels of pseudopterosin. Methods for producing pseudopterosins that include the steps of culturing algal cells under conditions that cause the cells to produce pseudopterosins and isolating the pseudopterosins so produced from the cells.

This application claims the benefit of U.S. application No. 60/482,095, entitled “PSEUDOPTEROSIN PRODUCTION,” filed on Jun. 24, 2003 which is hereby incorporated, herein by reference in its entirety.

### FIELD OF THE INVENTION

The invention generally relates to production of pseudopterosins from a renewable source and avoidance of destruction of coral reefs. In particular, alga which live in symbiosis with P. elisabethae can be replicated to large numbers in an in vitro culture, produce pseudopterosins at a level comparable to that found in naturally occurring coral.

### BACKGROUND

For decades, gorgonians (O. Gorgonacea, Ph. Cnidaria), commonly known as sea feathers, sea whips and sea fans, have been recognized as a rich source of chemically diverse compositions. Many of these compositions have been isolated by natural products chemists and are known to be useful for treating various diseases. Particularly useful among these are the pseudopterosins. Pseudopterosins were originally isolated from the marine soft coral, Pseudopterogorgia elisabethae, and are now of significant commercial importance because of their anti-inflammatory and anti-proliferative properties (Look et al., Proc. Natl. Acad. Sci. USA. 83:6238-6240, 1986; Look et al., J. Org. Chem. 51:5140-5145, 1986; Look et al., Tetrahedron 43:3363-3370, 1987; Roussis et al., J. Org. Chem. 55:4916-4922, 1990). Because organic methods for synthesizing such molecules is far from being commercially practical, the predominant method of producing pseudopterosins is by isolation from harvested coral. Unfortunately, harvesting of coral has an undesirable impact on the environment.

In order to protect the coral reefs from destruction, a need in the art exists for production of pseudopterosins that is cheap, fast, reliable and does not depend on the harvesting of marine life.

### SUMMARY OF THE INVENTION

The present invention relates to the development of a method and system for producing pseudopterosins that does not require massive destruction of coral reefs. The invention relates to the discovery that certain alga which live in symbiosis with P. elisabethae can be replicated to large numbers in an in vitro culture. When cultured under the appropriate conditions, these alga make pseudopterosins at a level comparable to that found in naturally occurring coral. The invention thus allows for the production of pseudopterosins from a renewable resource. Using the method and system of the invention should significantly reduce the cost of making pseudopterosins, and should also avoid the destruction of coral reefs.

In a preferred embodiment, the invention provides a composition comprising isolated pseudopterosin-producing alga cells in a culture medium. Preferably, the pseudopterosin-producing alga is isolated from a host organism, such as, the genus Pseudopterogorgia. Also preferred is the host, Pseudopterogorgia elisabethae.

In another preferred embodiment, the isolated pseudopterosin-producing alga is of the genus Symbiodinium, preferably, a clade B genus. Preferred pseudopterosin-producing alga is Symbiodinium microadricum.

In another preferred embodiment, the culture medium of the composition is ASP-8A culture medium. Culturing of said cells is preferably in a 14-10 hour light-dark cycle. The light intensity of the illumination is about 30 up to 150 μmol photons·m⁻²·s⁻¹. Preferably, the composition is maintained at a temperature between 23° C. to 29° C.

In another preferred embodiment, the cultured pseudopterosin producing alga are free of contaminating microorganisms. Preferably, cultured pseudopterosin producing alga are determined to be free of any contaminating microorganisms by amplification of DNA from cultured alga, wherein amplification is conducted using any one the primers identified by any one of SEQ ID NO's: 1-4.

In another preferred embodiment, the composition produces about 100% up to 1000% more pseudopterosin as compared to an initial isolated pseudopterosin-producing alga. Preferably, pseudopterosin levels are measured by HPLC.

In another preferred embodiment, the composition comprising pseudopterosin-producing alga produce pseudopterosins A-L, preferably, pseudopterosins A-D are produced.

In another preferred embodiment, the invention provides a culture comprising a pseudopterosin-producing alga, wherein the alga is of the genus Symbiodinium, preferably, Symbiodinium microadricum.

In another preferred embodiment, the alga is isolated from a host of the genus Pseudopterogorgia, preferably, Pseudopterogorgia elisabethae.

In accordance with the invention, the culture medium comprising pseudopterosin-producing alga, is subjected to illumination using a 14-10 light-dark cycle. Preferably, the light intensity of the illumination is 30-150 μmol photons·m⁻²·s⁻¹ and the culture is maintained at a temperature between 23-29° C.

In another preferred embodiment, the invention provides a method for producing pseudopterosins from isolated pseudopterosin-producing alga cells in a culture medium. Preferably, the pseudopterosin-producing alga is isolated from a host organism, such as Pseudopterogorgia elisabethae. Also preferred are isolated pseudopterosin-producing alga, Symbiodinium microadricum.

In a preferred embodiment, the isolated pseudopterosin-producing alga is cultured in ASP-8A culture medium and are subjected to illumination using a 14-10 hour light-dark cycle, wherein, the light intensity of the illumination is about 30 up to 150 μmol photons·m⁻²·s⁻¹, and maintained at a temperature between 23° C. to 29° C. The cultured pseudopterosin producing alga are free of contaminating microorganisms as determined by PCR amplification of isolated DNA using primers identified by any one of SEQ ID NO's: 1-4.

In another preferred embodiment, the method for producing pseudopterosins comprising the cultured pseudopterosin-producing alga produce about 100% up to 1000% more pseudopterosin as compared to an initial isolated pseudopterosin-producing alga, as measured by, for example, HPLC. Preferred pseudopterosins produced by the method of the invention are pseudopterosins A-D.

Other aspects of the invention are described infra.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting. Other features and advantages of the invention will be apparent from the following detailed description, and from the claims.

### BRIEF DESCRIPTION OF THE DRAWINGS

FIG. 1 is a graph displaying the amount of pseudopterosins produced by the algal cells according to the invention, at 4 time points during a 42 day culture.

FIG. 2 is a 2.5% agarose gel showing the restriction fragment length polymorphism (RFLP) analysis of initially isolated algal cells as well as that of algal cells that had been cultured in vitro for 8 weeks. Lane 1—1 kB ladder, Lane 2—Taq 1 digest of cultured alga 8 weeks after inoculation, Lane 4—1 kB ladder, Lane 5—DPN II digest of the initial algal isolate, Lane 6—DPN II digest of cultured alga 8 weeks after inoculation, Lane 7—1 kB ladder.

FIG. 3 is a 1.2% agarose gel displaying the ITS region analysis of initially isolated algal cells as well as that of algal cells that had been cultured in vitro for 26 weeks. Lane 1—1 kB ladder, Lane 3—negative control, Lane 6—initial alga isolate, Lane 9—cultured alga 26 weeks after inoculation, Lane 12—cultured alga 8 weeks after inoculation.

### DETAILED DESCRIPTION OF THE INVENTION

The invention provides compositions producing pseudopterosins and methods for cultivating alga for the production of pseudopterosins in vitro. In particular, algal cells are isolated from a host, and then introduced into an in vitro culture under conditions that cause the cells to proliferate and produce pseudopterosins.

Definitions

Prior to setting forth the invention, it may be helpful to an understanding thereof to set forth definitions of certain terms that will be used hereinafter.

The term “DNA construct” and “vector” are used herein to mean a purified or isolated polynucleotide that has been artificially designed and which comprises at least two nucleotide sequences that are not found as contiguous nucleotide sequences in their natural environment.

As used herein, the term “administering a molecule to a cell” (e.g., an expression vector, nucleic acid, a delivery vehicle, agent, and the like) refers to transducing, transfecting, microinjecting, electroporating, or shooting, the cell with the molecule. In some aspects, molecules are introduced into a target cell by contacting the target cell with a delivery cell (e.g., by cell fusion or by lysing the delivery cell when it is in proximity to the target cell).

A cell has been “transformed”, “transduced”, or “transfected” by exogenous or heterologous nucleic acids when such nucleic acids have been introduced inside the cell. Transforming DNA may or may not be integrated (covalently linked) with chromosomal DNA making up the genome of the cell. In prokaryotes, yeast, and mammalian cells for example, the transforming DNA may be maintained on an episomal element, such as a plasmid. In a eukaryotic cell, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations (e.g., at least about 10).

As used interchangeably herein, the terms “oligonucleotides”, “polynucleotides”, and “nucleic acids” include RNA, DNA, or RNA/DNA hybrid sequences of more than one nucleotide in either single chain or duplex form. The term “nucleotide” as used herein as an adjective to describe molecules comprising RNA, DNA, or RNA/DNA hybrid sequences of any length in single-stranded or duplex form. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. Although the term “nucleotide” is also used herein to encompass “modified nucleotides” which comprise at least one modifications (a) an alternative linking group, (b) an analogous form of purine, (c) an analogous form of pyrimidine, or (d) an analogous sugar, all as described herein.

As used herein, “molecule” is used generically to encompass any vector, antibody, protein, pseudopterosin, pseudopterosin derivatives, drug and the like.

As used herein, the term “oligonucleotide” refers to a polynucleotide formed from naturally occurring bases and pentofuranosyl groups joined by native phosphodiester bonds. This term effectively refers to naturally occurring species or synthetic species formed from naturally occurring subunits or their close homologs. The term “oligonucleotide” may also refer to moieties which function similarly to naturally occurring oligonucleotides but which have non-naturally occurring portions. Thus, oligonucleotides may have altered sugar moieties or intersugar linkages. Exemplary among these are the phosphorothioate and other sulfur-containing species which are known for use in the art. In accordance with some preferred embodiments, at least some of the phosphodiester bonds of the oligonucleotide have been substituted with a structure which functions to enhance the ability of the compositions to penetrate into the region of cells where the RNA or DNA whose activity to be modulated is located. It is preferred that such substitutions comprise phosphorothioate bonds, methyl phosphonate bonds, or short chain alkyl or cycloalkyl structures. In accordance with other preferred embodiments, the phosphodiester bonds are substituted with other structures which are, at once, substantially non-ionic and non-chiral, or with structures which are chiral and enantiomerically specific. Persons of ordinary skill in the art will be able to select other linkages for use in practice of the invention.

Oligonucleotides may also include species which include at least some modified base forms. Thus, purines and pyrimidines other than those normally found in nature may be so employed. Similarly, modifications on the pentofuranosyl portion of the nucleotide subunits may also be effected, as long as the essential tenets of this invention are adhered to. Examples of such modifications are 2′-O-alkyl- and 2′-halogen-substituted nucleotides. Some specific examples of modifications at the 2′ position of sugar moieties which are useful in the present invention are OH, SH, SCH₃, F, OCH₃, OCN, O(CH₂)ₙNH2 or O(CH₂)ₙCH₃ where n is from 1 to about 10, and other substituents having similar properties.

“Optional” or “optionally” means that the subsequently described event or circumstance may, but need not, occur, and that the description includes instances where the event or circumstance occurs and instances in which it does not. For example, “heterocyclo group optionally mono- or di-substituted with an alkyl group” means that the alkyl may, but need not, be present, and the description includes situations where the heterocyclo group is mono- or disubstituted with an alkyl group and situations where the heterocyclo group is not substituted with the alkyl group.

Compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

“Diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The terms “patient” or “individual” are used interchangeably herein, and is meant a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

As used herein, “ameliorated” or “treatment” refers to a symptom which is approaches a normalized value, e.g., is less than 50% different from a normalized value, preferably is less than about 25% different from a normalized value, more preferably, is less than 10% different from a normalized value, and still more preferably, is not significantly different from a normalized value as determined using routine statistical tests.

As used herein, the term “safe and effective amount” or “therapeutic amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. For example, an amount effective to delay the growth of or to cause a cancer, either a sarcoma or lymphoma, or to shrink the cancer or prevent metastasis. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, a “pharmaceutical salt” include, but are not limited to, mineral or organic acid salts of basic residues such as amines; alkali or organic salts of acidic residues such as carboxylic acids. Preferably the salts are made using an organic or inorganic acid. These preferred acid salts are chlorides, bromides, sulfates, nitrates, phosphates, sulfonates, formates, tartrates, maleates, malates, citrates, benzoates, salicylates, ascorbates, and the like. The most preferred salt is the hydrochloride salt.

As used herein, “cancer” refers to all types of cancer or neoplasm or malignant tumors found in mammals, including, but not limited to: leukemias, lymphomas, melanomas, carcinomas and sarcomas. Examples of cancers are cancer of the brain, breast, pancreas, cervix, colon, head and neck, kidney, lung, non-small cell lung, melanoma, mesothelioma, ovary, sarcoma, stomach, uterus and Medulloblastoma.

Additional cancers which can be treated with pseudopterosin molecules according to the invention include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, and prostate cancer.

A “test amount” of a pseudopterosin refers to an amount of a pseudopterosin present in a sample being tested. A test amount can be either in absolute amount (e.g., μg/ml) or a relative amount (e.g., relative intensity of signals).

A “control amount” of a pseudopterosin can be any amount or a range of amount which is to be compared against a test amount of a pseudopterosin. A control amount can be either in absolute amount (e.g., μg/ml) or a relative amount (e.g., relative intensity of signals).

“Probe” refers to a device that is removably insertable into a gas phase ion spectrometer and comprises a substrate having a surface for presenting a pseudopterosin for detection. A probe can comprise a single substrate or a plurality of substrates.

“Substrate” or “probe substrate” refers to a solid phase onto which an adsorbent can be provided (e.g., by attachment, deposition, etc.).

“Adsorbent” refers to any material capable of adsorbing a pseudopterosin. The term “adsorbent” is used herein to refer both to a single material (“monoplex adsorbent”) (e.g., a compound or functional group) to which the pseudopterosin is exposed, and to a plurality of different,materials (“multiplex adsorbent”) to which the pseudopterosin is exposed. The adsorbent materials in a multiplex adsorbent are referred to as “adsorbent species.” For example, an addressable location on a probe substrate can comprise a multiplex adsorbent characterized by many different adsorbent species (e.g., anion exchange materials, metal chelators, or antibodies), having different binding characteristics. Substrate material itself can also contribute to adsorbing a pseudopterosin and may be considered part of an “adsorbent.”

“Adsorption” or “retention” refers to the detectable binding between an absorbent and a pseudopterosin either before or after washing with an eluant (selectivity threshold modifier) or a washing solution.

“Eluant” or “washing solution” refers to an agent that can be used to mediate adsorption of a pseudopterosin to an adsorbent. Eluants and washing solutions are also referred to as “selectivity threshold modifiers.” Eluants and washing solutions can be used to wash and remove unbound materials from the probe substrate surface.

“Gas phase ion spectrometer” refers to an apparatus that measures a parameter which can be translated into mass-to-charge ratios of ions formed when a sample is volatilized and ionized. Generally ions of interest bear a single charge, and mass-to-charge ratios are often simply referred to as mass. Gas phase ion spectrometers include, for example, mass spectrometers, ion mobility spectrometers, and total ion current measuring devices.

“Mass spectrometer” refers to a gas phase ion spectrometer that includes an inlet system, an ionization source, an ion optic assembly, a mass analyzer, and a detector.

“Laser desorption mass spectrometer” refers to a mass spectrometer which uses laser as means to desorb, volatilize, and ionize an analyte.

“Detect” refers to identifying the presence, absence or amount of the object to be detected.

“Detectable moiety” or a “label” refers to a composition detectable by spectroscopic, photochemical, biochemical, immunochemical, or chemical means. The detectable moiety often generates a measurable signal, such as a radioactive, chromogenic, or fluorescent signal, that can be used to quantify the amount of bound detectable moiety in a sample. Quantitation of the signal is achieved by, e.g., scintillation counting, densitometry, or flow cytometry.

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha (α), gamma (γ), delta (δ), epsilon (ε), and mu (μ) heavy chain constant region genes, and the myriad immunoglobulin variable region genes. Antibodies exist, e.g., as intact immunoglobulins or as a number of well characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH₁, CH₂ and CH₃, but does not include the heavy chain variable region.

“Immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or “specifically (or selectively) immunoreactive with,” when referring to a pseudopterosin, refers to a binding reaction that is determinative of the presence of the pseudopterosin in a heterogeneous population of pseudopterosins and other biologics. Thus, under designated immunoassay conditions, the specified antibodies bind to a particular pseudopterosin at least two times the background and do not substantially bind in a significant amount to other pseudopterosins present in the sample. Specific binding to an antibody under such conditions may require an antibody that is selected for its specificity for a particular pseudopterosin. For example, polyclonal antibodies raised to pseudopterosin “X” from specific species such as rat, mouse, or human can be selected to obtain only those polyclonal antibodies that are specifically immunoreactive with pseudopterosin “X” and not with other pseudopterosins. A variety of immunoassay formats may be used to select antibodies specifically immunoreactive with a particular pseudopterosin. For example, solid-phase ELISA immunoassays (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

“Energy absorbing molecule” or “EAM” refers to a molecule that absorbs energy from an ionization source in a mass spectrometer thereby aiding desorption of analyte, such as a pseudopterosin, from a probe surface. Depending on the size and nature of the analyte, the energy absorbing molecule can be optionally used. Energy absorbing molecules used in MALDI are frequently referred to as “matrix.” Cinnamic acid derivatives, sinapinic acid (“SPA”), cyano hydroxy cinnamic acid (“CHCA”) and dihydroxybenzoic acid are frequently used as energy absorbing molecules in laser desorption of bioorganic molecules.

“Sample” is used herein in its broadest sense. A sample comprising pseudopterosin polynucleotides, polypeptides, peptides, and the like may comprise a soluble fraction of a cell preparation, or media in which cells were grown; a chromosome, an organelle, or membrane isolated or extracted from a cell; genomic DNA, RNA, or cDNA, in solution or bound to a substrate, a cell, and the like.

“Substantially purified” refers to nucleic acid molecules or pseudopterosins that are removed from their natural environment and are isolated or separated, and are at least about 60% free, preferably about 75% free, and most preferably about 90% free, from other components with which they are naturally associated.

“Substrate” refers to any rigid or semi-rigid support to which nucleic acid molecules or pseudopterosins are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

In a preferred embodiment, the invention provides a composition comprising isolated pseudopterosin-producing alga cells in a culture medium. Preferably, the pseudopterosin-producing alga is isolated from a host organism, such as, the genus Pseudopterogorgia. Also preferred is the host, Pseudopterogorgia elisabethae. Preferred pseudopterosin-producing alga is Symbiodinium microadricum.

In another preferred embodiment, the invention provides a method for producing pseudopterosins from isolated pseudopterosin-producing alga cells in a culture medium. Preferably, the pseudopterosin-producing alga is isolated from a host organism, such as Pseudopterogorgia elisabethae. Also preferred are isolated pseudopterosin-producing alga, Symbiodinium microadricum.

In another preferred embodiment, the method for producing pseudopterosins comprising the cultured pseudopterosin-producing alga produce about 100% up to 1000% more pseudopterosin as compared to an initial isolated pseudopterosin-producing alga, as measured by, for example, HPLC. Preferred pseudopterosins produced by the method of the invention are pseudopterosins A-D.

Isolation of Algal Cells

In a preferred embodiment, algal cells are isolated from the natural environment. For example, in the marine environment, symbiotic alga often form an association with a variety of marine organisms. Thus, alga may be obtained by harvesting from marine organisms such as Aiptasia, Anthopleura, Bartholomea, Cassiopeia, Condylactis, Corbulifera, Corculum, Dichotomia, Discosoma, Gorgonia, Heliopora, Hippopus, Lebrunia, Linuche, Mastigias, Meandrina, Montastraea, Montipora, Oculina, Pocillopora, Rhodactis, Stylophora, Tridacna, and Zoanthus. Marine organism hosts can be of the class Octocorallia that can include West Indian octocorals of the families Briareidae, Plexauridae, Gorgoniidae, Briarium, Erythropodium, Eunicea, Gorgonia, Leptogorgia, Muricea, Phyllogorgia, Plexaura, Plexaurella, Pseudoplexaura, Pseudopterogorgia, Pterogorgia. A preferred alga of the invention is one isolated from Pseudopterogorgia elisabethae because, as described below, these can produce high levels of pseudopterosins when cultured under appropriate conditions. A P. elisabethae organism useful according to the subject invention can be obtained in deeper waters ranging from about 30 to 100 feet in the Bahama Islands, e.g., Grand Bahama Island, Rum Cay, or Egg Cay. Freshly collected organisms can be stored frozen, e.g., in liquid nitrogen. Taxonomic identification can be confirmed by conducting thin la yer chromatography.

In another preferred embodiment, the taxonomic identification of the coral can be identified by PCR. For example, the isolated DNA from the initial isolate of flash frozen P. elisabethae which corresponds to the same sample in culture is subjected to PCR. SS5 and SS3Z zooxanthallae-specific primers were used for genomic and taxonomic identification. The SS5 primer is identified by 5′ggttgatcctgccagtagtcatatgcttg-3′ (SEQ ID NO: 1) and the SS3Z primer is identified by 5′-agcactgcgtcagtccgaataaatcaccgg-3′ (SEQ ID NO: 2). The PCR products were separated on a 0.8% agarose gel and stained with ethidium bromide. The DNA annealed between 1.65 and 2.0 kB and the specific product corresponded to 1.65 kB. The process was repeated for algal cells (which had been in culture for 8 weeks) and both products were excised from the gel and stored at −20° C.

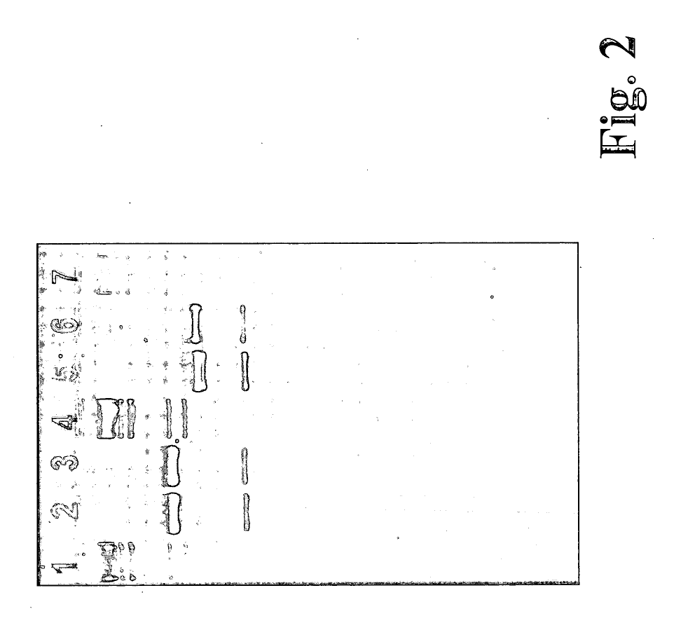

In another embodiment, isolated DNA from the coral and isolated pseudopterosin producing cultured alga are subjected to enzymatic digestion to confirm that the said alga were homogenous and not contaminated with other microorganisms. Preferably, the amplified DNA is subjected to RFLP analysis and illustrative results are shown in FIG. 2.

In another preferred embodiment, alga include those that produce a pseudopterosin, seco-pseudopterosin, or pseudopterosin or seco-pseudopterosin biosynthetic intermediate. For example, the alga can be a dinoflagellate such as an alga of the genus Symbiodinium. Examples of Symbiodinium species include S. kawagutii, S. goreaui, S. muscatinei, S. pulchrorum, S. bermudense, S. californium, S. microadriatiucum, S. pilosum, S. meandrinae, S. corculorum, and S. linucheae. S. microadricum is preferred for use in the invention because, as shown herein, it is capable of producing pseudopterosins at high levels in in vitro culture. As not all clades of S. microadricum make high levels of pseudopterosins under the culture conditions described below, preferred clade of S. microadricum is clade B.

In accordance with the invention, pseudopterosins produced by the cultured alga include but not limited to, for example, pseudopterosins A-D, which were the first compositions of this class to be isolated (Look et al., J. Org. Chem. 51:5140 (1986); pseudopterosins E-L. The sugar moiety in pseudopterosin E and F (Roussiss et al., J. Org. Chem. 55:4916 (1990)) is attached to the phenol at carbon 10 of the aglycon instead of the phenol at carbon 9, as in pseudopterosins A-D. In pseudopterosins G-J, the stereochemical configuration at carbon 7 of the aglycon is inverted with respect to pseudopterosins A-D. The aglycon of pseudopterosins K and L is enantiomeric to the aglycon of pseudopterosins A-D. Pseudopterosin derivatives are also useful for the methods described herein.

As used herein, a “pseudopterosin derivative” is a pseudopterosin in which the aglycon and/or the monosaccharide substructures are chemically modified and which promotes wound healing. Examples of pseudopterosin derivatives include pseudopterosins in which-the free phenol of the aglycon is alkylated or acylated and are referred to herein as “pseudopterosin alkyl ethers” or “acylated pseudopterosins”, respectively. Specific examples of pseudopterosin A derivatives include pseudopterosin A methyl ether (R2=—CH₃), pseudopterosin A 4-hydroxybutyl ether (R2=(—CH₂)₄ —OH)), pseudopterosin A pentyl ether (R2=—(CH₂)₄ —CH₃), pseudopterosin A acetamide ether (R2=—CH₂CO—NH.sub.2), and pseudopterosin A benzyl ether (R2=—CH₂—C₆H₅). Pseudopterosin derivatives also include pseudopterosins wherein the hydrocarbon side chain attached to carbon one is modified, for example by hydrogenation, or by oxidation of the 2-methyl-1-propene moiety to, for example, 1-keto-2-methyl-propane or 2-methyl-propeneoxide (Jacobs, et al., U.S. Pat. No. 4,849,410).

In another preferred embodiment, algal cells can be isolated from a marine organism by several conventional methods. For example, as described below alga cells are isolated from a host by grinding or blending the host in an aqueous medium such as filtered seawater, buffer, or culture medium. After the grinding or blending step, the algal cells are separated from the non-algal cells and debris by a suitable method, e.g., filtration and/or a density gradient separation. For example, a crude homogenate of coral can first be filtered through cheesecloth to remove cellular debris, and then subjected to repeated centrifugation (relatively dense algal cells collect in the pellet). Other purification steps that might be used include any method that separates cells based on cell size, density, surface charge or hydrophobic surface properties. Methods that maximally preserve cell viability are preferred. Specific examples of such methods include density gradient separation of cells using centrifugal elutriation [such as on silica particles coated with polyvinylpyrrolidone (Percoll®) or a non-ionic synthetic polymer of sucrose (Ficoll®)], partitioning between aqueous two-phase systems, flow cytometry, antibody-based methods (including magnetic, column, and panning techniques), and free flow electrophoresis according to known procedures in the art such as those described e.g., in Cell Separation: A Practical Approach (Practical Approach Series, 193), eds. Fisher, Francis, and Rickwood, Oxford University Press, New York, 1998.

Culture of Algal Cells

Following the isolation of algal cells, the cells are rinsed with filtered seawater or another suitable aqueous solution to remove residue from the previous purification step. The viability of the cells can be examined utilizing any stain (e.g., Trypan Blue) or fluorescent dye which is ingested by the cell and gives a measure of the quantity of living cells present in the sample (see previous reference). The amount of viable cells is determined by observing a portion of the sample under a microscope and counting the number of viable cells manually or automatically using an instrument such as a flow cytometer. The cell viability can also be evaluated using cell viability/proliferation assays known in the art. The cells in each sample are aliquoted into cell culture media based upon the number of surviving cells and grown in culture under culture conditions.

Various media and growth conditions can be utilized for culturing algal cells. See, e.g., Rowan et al., Proc. Natl . Aca. Sci. U S A. 89:3639-43, 1992, and references cited therein. Examples of culture medium that can support algal cell cultures include ASP-8A (Provasoli et al., Arch. Microbiol. 25: 392-428, 1957), Guillard's F/2 (Sigma), and Prov 50 (Guillard. Culture of phytoplankton for feeding marine invertebrates, In Smith and Chanley, (eds.) Culture of Marine Invertebrate Animals, Plenum Press, New York, 1975, pp 26-60; Guillard et al., Can. J. Microbiol. 8: 229-239, 1962; Provasoli, et al. Arch. Microbiol. 25: 392-428, 1957). Media can be supplemented with various nutrients, metals, and other additives (e.g., antibiotics) known in the art or described in the references herein.

In preferred embodiments, culture conditions important for algal cell growth include light intensity, illumination cycle, and temperature. For example, conditions for the culture of the alga of the invention include about a 14-10 hour light-dark cycle, a light intensity of about 30 to about 150 μmol photons·m⁻²·s⁻¹ and a temperature between 23-29° C.

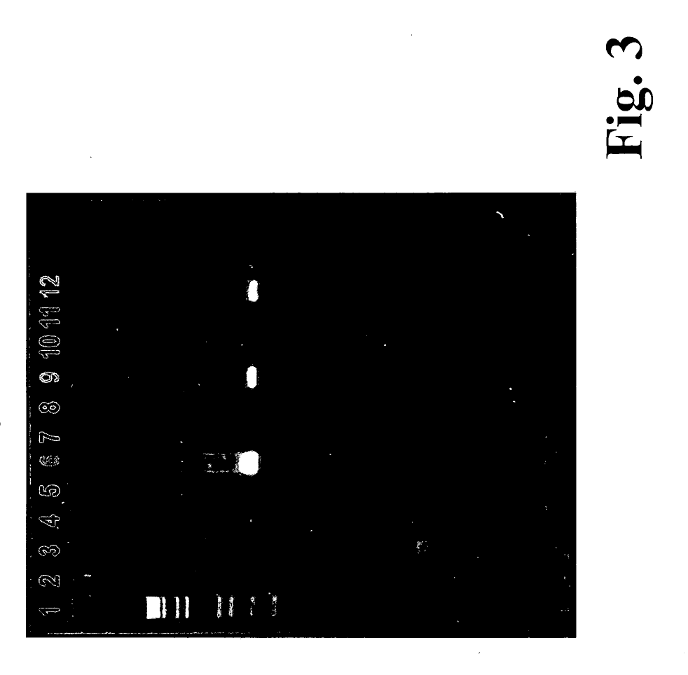

In another preferred embodiment, the pseudopterosin producing cultured alga cells are subjected to genetic analysis to confirm that the said cultured cells are clones of the original isolated pseudopterosin producing algal cell. Preferably, DNA is isolated from the cultured alga cells and subjected to PCR using primers specific for the ITS region (ITS ss5 and ITS ss3). The ITS ss5 primer is identified by 5′-gcatcgatgaagaacgcagc-3′ (SEQ ID NO: 3) and the ITS ss3 primer is identified by 5′gctgcgttcttcagcgat-3′ (SEQ ID. NO: 4). Illustrative results are shown in FIG. 3, confirming that the phylogeny of the cells in culture 26 weeks after inoculation was congruent with the phylogeny of the initial algal isolate.

Purification and Quantification of Pseudopterosins

The pseudopterosins present in the cells can be quantified by conventional methods, e.g., using high-performance liquid chromatography (HPLC). The pseudopterosins of interest can include both pseudopterosins and seco-pseudopterosins as well as intermediates involved in the biosynthesis of these classes of compositions. The pseudopterosins can be purified from the algal cells according to known procedures such as those described by Look et al., Proc. Natl. Acad. Sci. USA. 83:6238-6240, 1986; Look et al., J. Org. Chem. 51:5140-5145, 1986; Look et al., Tetrahedron 43:3363-3370, 1987; Roussis et al., J. Org. Chem. 55:4916-4922, 1990; and U.S. Pat. Nos. 4,849,410, 4,745,104, and 5,624,911. In addition to these purification methods, other chromatographic techniques such as ion-exchange, size-exclusion, thin-layer, supercritical fluid, capillary electrophoresis, and gas chromatography could be used in the separation and analysis of pseudopterosins from the algal cells.

Other procedures for isolation and purification of pseudopterosins produced by the cultured alga of the invention can also be used. Typically, preparation involves fractionation of the sample and collection of fractions determined to contain the pseudopterosins. Methods of pre-fractionation include, for example, size exclusion chromatography, ion exchange chromatography, heparin chromatography, affinity chromatography, sequential extraction, gel electrophoresis and liquid chromatography. The analytes also may be modified prior to detection. These methods are useful to simplify the sample for further analysis. For example, it can be useful to remove high abundance pseudopterosins before analysis.

In one embodiment, a sample can be pre-fractionated according to size of pseudopterosins in a sample using size exclusion chromatography. For a biological sample wherein the amount of sample available is small, preferably a size selection spin column is used. In general, the first fraction that is eluted from the column (“fraction 1”) has the highest percentage of high molecular weight pseudopterosins; fraction 2 has a lower percentage of high molecular weight pseudopterosins; fraction 3 has even a lower percentage of high molecular weight pseudopterosins; fraction 4 has the lowest amount of large pseudopterosins; and so on. Each fraction can then be analyzed by immunoassays, gas phase ion spectrometry, and the like, for the detection of pseudopterosins.

In another embodiment, a sample can be pre-fractionated by anion exchange chromatography. Anion exchange chromatography allows pre-fractionation of the pseudopterosins in a sample roughly according to their charge characteristics. For example, a Q anion-exchange resin can be used (e.g., Q HyperD F, Biosepra), and a sample can be sequentially eluted with eluants having different pH's. Anion exchange chromatography allows separation of pseudopterosins in a sample that are more negatively charged from other types of pseudopterosins. Pseudopterosins that are eluted with an eluant having a high pH is likely to be weakly negatively charged, and a fraction that is eluted with an eluant having a low pH is likely to be strongly negatively charged. Thus, in addition to reducing complexity of a sample, anion exchange chromatography separates pseudopterosins according to their binding characteristics.

In yet another embodiment, a sample can be fractionated using a sequential extraction protocol. In sequential extraction, a sample is exposed to a series of adsorbents to extract different types of pseudopterosins from a sample. For example, a sample is applied to a first adsorbent to extract certain pseudopterosins, and an eluant containing non-adsorbent pseudopterosins (i.e., pseudopterosins that did not bind to the first adsorbent) is collected. Then, the fraction is exposed to a second adsorbent. This further extracts various pseudopterosins from the fraction. This second fraction is then exposed to a third adsorbent, and so on.

Any suitable materials and methods can be used to perform sequential extraction of a sample. For example, a series of spin columns comprising different adsorbents can be used. In another example, a multi-well comprising different adsorbents at its bottom can be used. In another example, sequential extraction can be performed on a probe adapted for use in a gas phase ion spectrometer, wherein the probe surface comprises adsorbents for binding pseudopterosins. In this embodiment, the sample is applied to a first adsorbent on the probe, which is subsequently washed with an eluant. Pseudopterosins that do not bind to the first adsorbent are removed with an eluant. The pseudopterosins that are in the fraction can be applied to a second adsorbent on the probe, and so forth. The advantage of performing sequential extraction on a gas phase ion spectrometer probe is that pseudopterosins that bind to various adsorbents at every stage of the sequential extraction protocol can be analyzed directly using a gas phase ion spectrometer.

In yet another embodiment, pseudopterosins in a sample can be separated by high-resolution electrophoresis, e.g., one or two-dimensional gel electrophoresis. A fraction containing a pseudopterosin can be isolated and further analyzed by gas phase ion spectrometry. Preferably, two-dimensional gel electrophoresis is used to generate two-dimensional array of spots of pseudopterosins, including one or more pseudopterosins. See, e.g., Jungblut and Thiede, Mass Spectr. Rev. 16:145-162 (1997).

The two-dimensional gel electrophoresis can be performed using methods known in the art. See, e.g., Deutscher ed., Methods In Enzymology vol. 182. Typically, pseudopterosins in a sample are separated by, e.g., isoelectric focusing, during which pseudopterosins in a sample are separated in a pH gradient until they reach a spot where their net charge is zero (i.e., isoelectric point). This first separation step results in one-dimensional array of pseudopterosins. The pseudopterosins in one dimensional array is further separated using a technique generally distinct from that used in the first separation step. For example, in the second dimension, pseudopterosins separated by isoelectric focusing are further separated using a polyacrylamide gel, such as polyacrylamide gel electrophoresis in the presence of sodium dodecyl sulfate (SDS-PAGE). SDS-PAGE gel allows further separation based on molecular mass of pseudopterosins.

Prior to gas phase ion spectrometry analysis, it may be desirable to cleave pseudopterosins in the spot into smaller fragments using cleaving reagents, such as proteases (e.g., trypsin). The digestion of pseudopterosins into small fragments provides a mass fingerprint of the pseudopterosins in the spot, which can be used to determine the identity of pseudopterosins if desired.

In yet another embodiment, high performance liquid chromatography (HPLC) can be used to separate a mixture of pseudopterosins in a sample based on their different physical properties, such as polarity, charge and size. HPLC instruments typically consist of a reservoir of mobile phase, a pump, an injector, a separation column, and a detector. Pseudopterosins in a sample are separated by injecting an aliquot of the sample onto the column. Different pseudopterosins in the mixture pass through the column at different rates due to differences in their partitioning behavior between the mobile liquid phase and the stationary phase. A fraction that corresponds to the molecular weight and/or physical properties of one or more pseudopterosins can be collected. The fraction can then be analyzed by gas phase ion spectrometry to detect pseudopterosins.

After preparation, pseudopterosins in a sample are typically captured on a substrate for detection. A capture reagent or adsorbent is attached to the surface of the substrate. Frequently, the surface comprises a plurality of addressable locations, each of which location has the capture reagent bound there. The capture reagent can be a chromatographic material, such as an anion exchange material or a hydrophilic material. Captured pseudopterosins can be detected, for example, by mass spectrometry, fluorescence, surface plasmon resonance, ellipsometry and atomic force microscopy. Mass spectrometry, and particularly SELDI mass spectrometry, is a particularly useful method for detection of the pseudopterosins of this invention.

Preferably, a laser desorption time-of-flight mass spectrometer is used in embodiments of the invention. In laser desorption mass spectrometry, a substrate or a probe comprising pseudopterosins is introduced into an inlet system. The pseudopterosins are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of pseudopterosins of specific mass to charge ratio.

Matrix-assisted laser desorption/ionization mass spectrometry, or MALDI-MS, is a method of mass spectrometry that involves the use of an energy absorbing molecule, frequently called a matrix, for desorbing pseudopterosins intact from a probe surface. MALDI is described, for example, in U.S. Pat. No. 5,118,937 (Hillenkamp et al.) and U.S. Pat. No. 5,045,694 (Beavis and Chait). In MALDI-MS the sample is typically mixed with a matrix material and placed on the surface of an inert probe. Exemplary energy absorbing molecules include cinnamic acid derivatives, sinapinic acid (“SPA”), cyano hydroxy cinnamic acid (“CHCA”) and dihydroxybenzoic acid. Other suitable energy absorbing molecules are known to those skilled in this art. The matrix dries, forming crystals that encapsulate the analyte molecules. Then the analyte molecules are detected by laser desorption/ionization mass spectrometry. MALDI-MS is useful for detecting the pseudopterosins of this invention if the complexity of a sample has been substantially reduced using the preparation methods described above.

Surface-enhanced laser desorption/ionization mass spectrometry, or SELDI-MS represents an improvement over MALDI for the fractionation and detection of biomolecules, such as pseudopterosins, in complex mixtures. SELDI is a method of mass spectrometry in which biomolecules, such as pseudopterosins, are captured on the surface of a pseudopterosin biochip using capture reagents that are bound there. Typically, non-bound molecules are washed from the probe surface before interrogation. SELDI is described, for example,. in: U.S. Pat. No. 5,719,060 (“Method and Apparatus for Desorption and Ionization of Analytes,” Hutchens and Yip, Feb. 17, 1998,) U.S. Pat. No. 6,225,047 (“Use of Retentate Chromatography to Generate Difference Maps,” Hutchens and Yip, May 1, 2001) and Weinberger et al., “Time-of-flight mass spectrometry,” in Encyclopedia of Analytical Chemistry, R. A. Meyers, ed., pp 11915-11918 John Wiley & Sons Chichesher, 2000.

Pseudopterosins on the substrate surface can be desorbed and ionized using gas phase ion spectrometry. Any suitable gas phase ion spectrometers can be used as long as it allows pseudopterosins on the substrate to be resolved. Preferably, gas phase ion spectrometers allow quantitation of pseudopterosins.

In one embodiment, a gas phase ion spectrometer is a mass spectrometer. In a typical mass spectrometer, a substrate or a probe comprising pseudopterosins on its surface is introduced into an inlet system of the mass spectrometer. The pseudopterosins are then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of the presence of pseudopterosins or other substances will typically involve detection of signal intensity. This, in turn, can reflect the quantity and character of pseudopterosins bound to the substrate. Any of the components of a mass spectrometer (e.g., a desorption source, a mass analyzer, a detector, etc.) can be combined with other suitable components described herein or others known in the art in embodiments of the invention.

In another embodiment, an immunoassay can be used to detect and analyze pseudopterosins in a sample. This method comprises: (a) providing an antibody that specifically binds to a pseudopterosin; (b) contacting a sample with the antibody; and (c) detecting the presence of a complex of the antibody bound to the pseudopterosin in the sample.

To prepare an antibody that specifically binds to a pseudopterosin, purified pseudopterosins or their nucleic acid sequences can be used. Nucleic acid and amino acid sequences for pseudopterosins can be obtained by further characterization of these pseudopterosins.

Using the purified pseudopterosins or their nucleic acid sequences, antibodies that specifically bind to a pseudopterosin can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). Such techniques include, but are not limited to, antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

After the antibody is provided, a pseudopterosin can be detected and/or quantified using any of suitable immunological binding assays known in the art (see, e.g., U.S. Pat. Nos. 4,366,241; 4,376,110; 4,517,288; and 4,837,168). Useful assays include, for example, an enzyme immune assay (EIA) such as enzyme-linked immunosorbent assay (ELISA), a radioimmune assay (RIA), a Western blot assay, or a slot blot assay. These methods are also described in, e.g., Methods in Cell Biology: Antibodies in Cell Biology, volume 37 (Asai, ed. 1993); Basic and Clinical Immunology (Stites & Terr, eds., 7th ed. 1991); and Harlow & Lane, supra.

Generally, a sample obtained from a subject can be contacted with the antibody that specifically binds the pseudopterosin. Optionally, the antibody can be fixed to a solid support to facilitate washing and subsequent isolation of the complex, prior to contacting the antibody with a sample. Examples of solid supports include glass or plastic in the form of, e.g., a microtiter plate, a stick, a bead, or a microbead. Antibodies can also be attached to a probe substrate

After incubating the sample with antibodies, the mixture is washed and the antibody-pseudopterosin complex formed can be detected. This can be accomplished by incubating the washed mixture with a detection reagent. This detection reagent may be, e.g., a second antibody which is labeled with a detectable label. Exemplary detectable labels include magnetic beads (e.g., DYNABEADS™), fluorescent dyes, radiolabels, enzymes (e.g., horse radish peroxide, alkaline phosphatase and others commonly used in an ELISA), and calorimetric labels such as colloidal gold or colored glass or plastic beads. Alternatively, the pseudopterosin in the sample can be detected using an indirect assay, wherein, for example, a second, labeled antibody is used to detect bound pseudopterosin-specific antibody, and/or in a competition or inhibition assay wherein, for example, a monoclonal antibody which binds to a distinct epitope of the pseudopterosin is incubated simultaneously with the mixture.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, pseudopterosin, volume of solution, concentrations and the like. Usually the assays will be carried out at ambient temperature, although they can be conducted over a range of temperatures, such as 10° C. to 40° C.

Immunoassays can be used to determine presence or absence of a pseudopterosin in a sample as well as the quantity of a pseudopterosin in a sample. First, a test amount of a pseudopterosin in a sample can be detected using the immunoassay methods described above. If a pseudopterosin is present in the sample, it will form an antibody-pseudopterosin complex with an antibody that specifically binds the pseudopterosin under suitable incubation conditions described above. The amount of an antibody-pseudopterosin complex can be determined by comparing to a standard. A standard can be, e.g., a known compound or another pseudopterosin known to be present in a sample. As noted above, the test amount of pseudopterosin need not be measured in absolute units, as long as the unit of measurement can be compared to a control.

Data generated by desorption and detection of pseudopterosins can be analyzed using any suitable means. In one embodiment, data is analyzed with the use of a programmable digital computer. The computer program generally contains a readable medium that stores codes. Certain code can be devoted to memory that includes the location of each feature on a probe, the identity of the adsorbent at that feature and the elution conditions used to wash the adsorbent. The computer also contains code that receives as input, data on the strength of the signal at various molecular masses received from a particular addressable location on the probe. This data can indicate the number of pseudopterosins detected, including the strength of the signal generated by each pseudopterosin.

Data analysis can include the steps of determining signal strength (e.g., height of peaks) of a pseudopterosin detected and removing “outliers” (data deviating from a predetermined statistical distribution). The observed peaks can be normalized, a process whereby the height of each peak relative to some reference is calculated. For example, a reference can be background noise generated by instrument and chemicals (e.g., energy absorbing molecule) which is set as zero in the scale. Then the signal strength detected for each pseudopterosin or other biomolecules can be displayed in the form of relative intensities in the scale desired (e.g., 100). Alternatively, a standard (e.g., a serum pseudopterosin) may be admitted with the sample so that a peak from the standard can be used as a reference to calculate relative intensities of the signals observed for each pseudopterosin or other pseudopterosins detected.

The computer can transform the resulting data into various formats for displaying. In one format, referred to as “spectrum view or retentate map,” a standard spectral view can be displayed, wherein the view depicts the quantity of pseudopterosin reaching the detector at each particular molecular weight. In another format, referred to as “peak map,” only the peak height and mass information are retained from the spectrum view, yielding a cleaner image and enabling pseudopterosins with nearly identical molecular weights to be more easily seen. In yet another format, referred to as “gel view,” each mass from the peak view can be converted into a grayscale image based on the height of each peak, resulting in an appearance similar to bands on electrophoretic gels. In yet another format, referred to as “3-D overlays,” several spectra can be overlaid to study subtle changes in relative peak heights. In yet another format, referred to as “difference map view,” two or more spectra can be compared, conveniently highlighting unique pseudopterosins and pseudopterosins which are up- or down-regulated between samples. Pseudopterosin profiles (spectra) from any two samples may be compared visually. In yet another format, Spotfire Scatter Plot can be used, wherein pseudopterosins that are detected are plotted as a dot in a plot, wherein one axis of the plot represents the apparent molecular mass of the pseudopterosins detected and another axis represents the signal intensity of pseudopterosins detected. For each sample, pseudopterosins that are detected and the amount of pseudopterosins present in the sample can be saved in a computer readable medium. This data can then be compared to a control (e.g., a profile or quantity of pseudopterosins detected in control).

Therapeutic Uses

The compositions of the present invention can be used to treat cancers and to promote the healing of a wide variety of wounds, including wounds to external epithelial tissue, internal epithelial tissue, dental tissue and eye tissue. Wounds to external epithelial tissue are preferred and are of several types: excisional, burns, dermal skin ulcers, lesions due to dermatological diseases, and atopic dermititus due to immediate type hypersensitivity.

“Excisional wounds” include tears, cuts, punctures or lacerations in the epithelial layer of the skin and may extend into the dermal layer and even into subcutaneous fat and beyond. Excisional wounds can result from surgical procedures or from accidental penetration of the skin.

“Burn wounds” refer to cases where large surface areas of skin have been removed or lost from an individual. The loss of skin refers to the epidermal layer, and usually includes the dermal layer. Wounds of this type include cases where part of the dermis has been lost or where the wound penetrates to subcutaneous fat and beyond. A burn wound can also result when an individual's skin is exposed to a chemical agent. Typically, burn wounds result when a large area of an individual's skin is exposed to heat, such as in a fire. As used herein, burn wounds also include cases where large areas of the skin have been removed through abrasion or surgically, e.g. to remove a skin cancer or to provide a skin autograft.

“Dermal skin ulcers” refer to lesions on the skin caused by superficial loss of tissue, usually with inflammation. Dermal skin ulcers which can be treated by the method of the present invention include decubitus ulcers, diabetic ulcers, venous stasis ulcers and arterial ulcers. Decubitus wounds refer to chronic ulcers that result from pressure applied to areas of the skin for extended periods of time. Wounds of this type are often called bedsores or pressure sores. Venous stasis ulcers result from the stagnation of blood or other fluids from defective veins. Arterial ulcers refer to necrotic skin in the area around arteries having poor blood flow.

“Dental tissue” refers to tissue in the mouth which is similar to epithelial tissue, for example gum tissue. Thus, the compositions of the present invention are useful for treating periodontal disease. “Internal epithelial tissue” refers to tissue inside the body which has characteristics similar to the epidermal layer in the skin. Examples include the lining of the intestine. Consequently, the compositions of present invention are useful for promoting the healing of certain internal wounds, for example wounds resulting from surgery. A “wound to eye tissue” refers to severe dry eye syndrome, corneal ulcers and abrasions and ophthalmic surgical wounds.

Wounds caused by dermatological diseases include lesions resulting from autoimmune disorders such as psoriasis. “Atopic dermititis” refers to skin trauma resulting from allergies associated with an immune response caused by allergens such as pollens, foods, dander, insect venoms and plant toxins.

A compound which “promotes the healing of a wound” results in the wound healing more quickly as a result of the treatment than a similar wound heals in the absence of the treatment. “Promotion of wound healing” can also mean that the compositions causes the proliferation and growth of keratinocytes, fibroblasts and endothelial cells, or that the wound heals with less scarring, less wound contraction, less collagen deposition and more superficial surface area. In certain instances, “promotion of wound healing” can also mean that certain methods of wound healing have improved success rates, (e.g. the take rates of skin grafts,) when used together with the compositions of the present invention. These compositions can promote the healing of wounds on humans and animals such as dogs, farm animals, guinea pigs, cats and the like.

The composition used in the present invention to promote wound healing comprises an effective wound healing amount of a pseudopterosin or a pseudopterosin derivative produced by the methods detailed in the Examples which follow.

The pseudopterosin compositions produced in accordance with the present invention are useful in the treatment of rheumatoid arthritis, osteoarthritis, rheumatic carditis, collagen and/or auto-immune diseases such as myasthenia gravis, allergic diseases, bronchial asthma and ocular and skin inflammatory diseases such as poison ivy. The compositions are also useful in treating proliferative diseases such a psoriasis. The compositions are useful in treating other skin diseases such as richen planus and pemphigus.

In other preferred embodiments, the compositions are used in treating leukemia type cancers. Leukemia type cancers such as acute lymphoblastic leukemia, acute myeloblastic leukemia, acute monoblastic leukemia, chronic lymphocytic leukemia and chronic granulocytic leukemia can be treated. Further, the compositions are expected to be useful against other types of cancers when used along or in combination with other anti-cancer drugs.

The compositions are also useful as adjuvant therapy associated with organ and tissue transplants and any neurological disease involving metabolism of nervous tissue phospholipid such as multiple sclerosis. Because of their selective antagonism of chemical irritation (i.e., PMA inflammation) pseudopterosin compositions can be useful in the treatment of insect bites, bee or wasp stings or any venom in which a major constituent is the enzyme phospholipase A₂. The compositions are potent non-narcotic analgesics and may be used to alleviate pain resulting from traumatic injury or acute progressive disease, such as post operative pain, burns, or other conditions involving a coincident inflammation.

Pharmaceutical Preparations

Also provided by the invention are pharmaceutical preparations of the subject compositions. The subject compositions can be incorporated into a variety of formulations for therapeutic administration. More particularly, the compositions of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. The formulations may be designed for administration via a number of different routes, including oral, buccal, rectal, parenteral, intraperitoneal, intradermal, transdermal, intracheal, etc., administration.

In pharmaceutical dosage forms, the subject compositions of the invention may be administered in the form of their pharmaceutically acceptable salts, or they may also be used alone or in appropriate association, as well as in combination, with other pharmaceutically active compositions. The following methods and excipients are merely exemplary and are in no way limiting.

For oral preparations, the subject compositions can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The subject compositions of the invention can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The compositions of the invention can be utilized in aerosol formulation to be administered via inhalation. The compositions of the present invention can be formulated into pressurized acceptable propellants such as dichlorodifluoromethane, propane, nitrogen and the like.

Furthermore, the subject compositions can be made into suppositories by mixing with a variety of bases such as emulsifying bases or water-soluble bases. The compositions of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

Depending on the patient and condition being treated and on the administration route, the subject compositions may be administered in dosages of, for example, from about 0.01 g to about 10 mg/kg body weight per day. The range is broad, since in general the efficacy of a therapeutic effect for different mammals varies widely with doses typically being 20, 30 or even 40 times smaller (per unit body weight) in man than in the rat. Similarly the mode of administration can have a large effect on dosage. Thus, for example, oral dosages may be ten times the injection dose. Higher doses may be used for localized routes of delivery.

A typical dosage may be a solution suitable for intravenous administration; a tablet taken from two to six times daily, or one time-release capsule or tablet taken once a day and containing a proportionally higher content of active ingredient, etc. The time-release effect may be obtained by capsule materials that dissolve at different pH values, by capsules that release slowly by osmotic pressure, or by any other known means of controlled release.

For use in the subject methods, the subject compositions may be formulated with other pharmaceutically active agents, including lipoxygenase-inhibiting agents.

Pharmaceutically acceptable excipients usable with the invention, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Those of skill in the art will readily appreciate that dose levels can vary as a finction of the specific compound, the severity of the symptoms and the susceptibility of the subject to side effects. Preferred dosages for a given compound are readily determinable by those of skill in the art by a variety of means.

Kits with unit doses of the subject compositions, usually in oral or injectable doses, are provided. In such kits, in addition to the containers containing the unit doses will be an informational package insert describing the use and attendant benefits of the drugs in treating pathological condition of interest. Preferred compositions and unit doses are those described herein above.

The present invention is further illustrated by the following specific examples. The examples are provided for illustration only and are not to be construed as limiting the scope or content of the invention in any way.

It is to be understood that while the invention has been described in conjunction with the preferred specific embodiments thereof, that the foregoing description as well as the examples which follow are intended to illustrate and not limit the scope of the invention. Other aspects, advantages and modifications within the scope of the invention will be apparent to those skilled in the art to which the invention pertains.

### EXAMPLES

### Example 1

Isolation of the Algal Cells

A coral sample, Pseudopterogorgia elisabethae, was obtained from Sweetings Cay, Bahamas at a depth of about 10-15 meters. Live coral was homogenized (in seawater filtered with a 0.22 μm filter) with either a homogenizer or a blender. The crude homogenate was filtered through cheesecloth to remove large skeletal parts. The homogenate was then centrifuged at 1,000×g for 10 min. Following centrifugation, the supematant was decanted and the algal cell pellet was resuspended in filtered seawater. This centrifugation process repeated at least 10 times.

The algal pellet was further purified using a discontinuous density gradient of colloidal silica coated with polyvinylpyrrolidone (Percoll®, Sigma). The Percoll® was diluted with filtered seawater and the gradient involved layering 10 mL of 30% on top of 10 mL of 70% Percoll®, on top of 5 mL of 100% Percoll®. The cell suspension (10 mL) was layered on top of the 30% layer and separated by centrifuging at 1000×g for 10 min. The algal cells trapped between the Percolle® layers were removed with a pipette. To remove any Percoll® residue, the purified alga was rinsed two additional times with filtered seawater.

### Example 2

Cell Viability Tests and Cell Culture

Purified algal cells were stained with Trypan Blue (Sigma) solution to assess the cell viability. Using a hemocytometer, the cells were observed under a microscope and counted. If the cells were brown in color were considered to be alive and if they were blue or clear in color, they were considered to be dead. The concentration of the cells that were deemed to be viable was adjusted and the cells (˜10⁵-10⁶) were aliquoted into ASP-8A media (Provasoli et al., Arch. Microbiol. 25: 392-428, 1957). Antibiotic mix was then added to 1% of the final concentration of the solution. Antibiotic mix consisted of preparing a stock solution of the following ingredients (solubilized in ASP-8A media unless otherwise noted): polymixin (0.9 mg/mL, solubilized in EtOH), streptomycin (33 mg/mL), penicillin-G (5.8 mg/mL), neomycin (20 mg/mL), tetracycline (2.5 mg/mL), chloramphenicol (10 mg/mL, solubilized in 95% EtOH). These ingredients were then added in the following amounts and diluted to 100 mL with ASP-8A media: polymixin =1 mL, streptomycin =10 mL, penicillin-G =2.24 mL, neomycin =1 mL, tetracycline =0.1 mL, and chloramphenicol =5 μL. The cultures were maintained between 23-29° C. on a 14-10 light-dark cycle and illuminated with wide spectrum bulbs at 30-150 μmol photons·m⁻²·s⁻¹(Ei).

### Example 3

Quantification of Pseudopterosins

Cells in culture (2×10⁵-3×10⁷) were dried on a lyophilizer, extracted with ethyl acetate, and the resulting dried crude extract partitioned between methanol/water (9:1). The methanol/water fraction was again partitioned between methanol/water (1:1) and methylene chloride. The resulting methylene chloride extract was analyzed using normal phase HPLC with a gradient elution (hexanes:ethyl acetate) of increasing ethyl acetate. The pseudopterosins (A-D) were identified by comparing the HPLC elution profile to a standard sample. Quantities of pseudopterosins (in mg) were determined from a calibration curve made using known amounts of pseudopterosins (A-D).

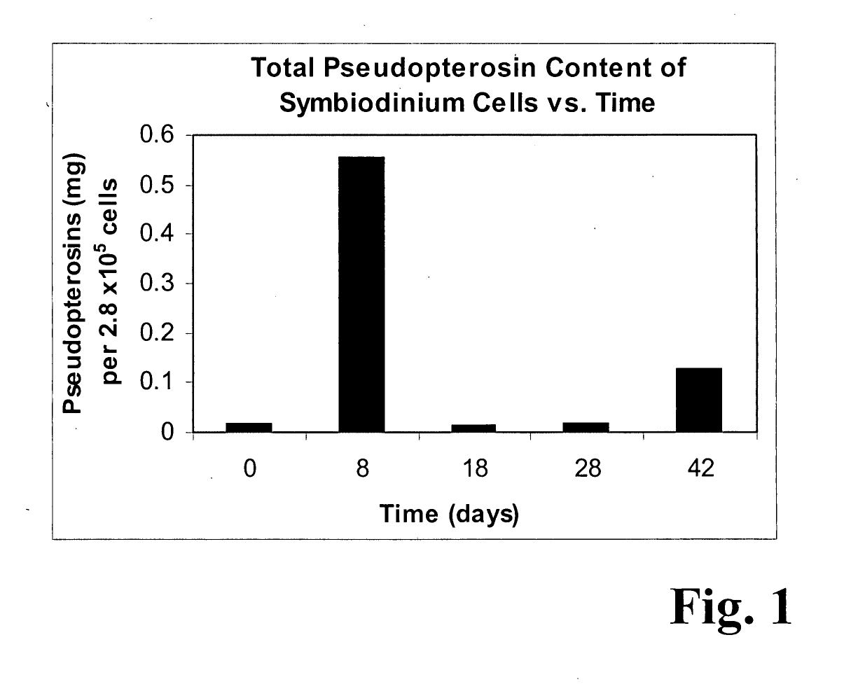

FIG. 1 is a graph displaying the amount of pseudopterosins present in the cells compared to the total amount of time the cells were in culture. The initial increase in the level of pseudopterosins per 2.8×10⁵ cells was typical due to the stress the cells endured from inoculation of the culture. Following the preliminary increase, the amount of pseudopterosins in the cells returned to a level comparable to the initial isolate. At day 42, the level of pseudopterosins present in the algal cells was more than 700% greater than that of the initial isolate indicating that the observed increase in pseudopterosin content was due to the production of pseudopterosins by the algal cells.

### Example 4

PCR Amplicification with Taq Polymerase and RFLP Analysis of Initial Isolate and Algal Cell Culture

The initial isolate of flash frozen P. elisabethae which corresponded to the same sample in culture was homogenized with liquid nitrogen using a mortar and pestle. The DNA was isolated using a DNA Plant Mini Kit for Isolation of DNA (Qiagen, Valencia, Calif.). PCR was performed using 10 μM SS5 and 5.6 μM SS3Z zooxanthallae-specific primers. The 10 μM SS5 primer used was 5′ ggttgatcctgccagtagtcatatgcttg-3′ and the 5.6 μM SS3Z primer used was 5′-agcactgcgtcagtccgaataaatcaccgg-3′. The PCR conditions utilized were: 92° C. for 3 min followed by 30 cycle at 80° C. for 5 min, 92° C. for 30 s, 52° C. for 40 s, 72° C. for 30 s, and then a final cycle at 72° C. for 5 min. The PCR products were separated on a 0.8% agarose gel and stained with ethidium bromide. The DNA annealed between 1.65 and 2.0 kB and the specific product corresponded to 1.65 kB. The process was repeated for algal cells (which had been in culture for 8 weeks) and both products were excised from the gel and stored at −20° C.

The PCR products from both the initial isolate and the cultured cells were digested with Taq 1 polymerase as well as a DPN II restriction enzyme. The Taq 1 polymerase digestion was conducted at 65° C. for 270 min and the DPN II digestion was performed at 37° C. for 240 min. The samples were separated on a 2.5% agarose gel and stained with ethidium bromide. FIG. 2 displays the agarose gel: Lane 1—1 kB ladder, Lane 2—Taq 1 digest of cultured alga 8 weeks after inoculation, Lane 4—1 kB ladder, Lane 5—DPN II digest of the initial algal isolate, Lane 6—DPN II digest of cultured alga 8 weeks after inoculation, Lane 7—1 kB ladder. The RFLP analysis results demonstrated that cells in culture 8 weeks after inoculation were still of the same clade (clade B) as the initial algal isolate (Toller et al., Biol. Bull. 201: 348-359, 2001; Santos, Taylor and Coffroth 2001 J. Phycol. 37, 900-912). Additionally, the results verified that the culture was homogenous and had not been overtaken by other microorganisms.

### Example 5

ITS Region Sequence Analysis Analysis of Initial Isolate and Algal Cell Culture

The DNA from the initial isolate as well as the cultured cells was isolated as described in Example 4. Primers specific for the ITS region (ITS ss5 and ITS ss3) were utilized for PCR. The ITS ss5 primer used was 5′-gcatcgatgaagaacgcagc-3′ and the ITS ss3 primer used was 5′gctgcgttcttcagcgat-3′. The PCR conditions were: 94° C. for 2 min followed by 30 cycle (at 94° C. for 0.4 min, 53° C. for 1 min, 72° C. for 1 min), and then a final cycle at 72° C. for 30 min. The PCR products were analyzed using a 1.2% agarose gel and stained with ethidium bromide. FIG. 3 displays the agarose gel: Lane 1—1 kB ladder, Lane 3—negative control, Lane 6—initial alga isolate, Lane 9—cultured alga 26 weeks after inoculation, Lane 12—cultured alga 8 weeks after inoculation. The results indicate that the phylogeny of the cells in culture 26 weeks after inoculation was congruent with the phylogeny of the initial algal isolate.

Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All references are incorporated in pertinent part by reference herein for the reasons cited in the above text.

1. A composition comprising:
a culture medium; and,
isolated pseudopterosin-producing alga cells.

2. The composition of claim 1, wherein the pseudopterosin-producing alga is isolated from a live marine host organism.

3. The composition of claim 2, wherein the pseudopterosin-producing alga is isolated from a host of the genus Pseudopterogorgia.

4. The composition of claim 3, wherein the pseudopterosin-producing alga is isolated from the host, Pseudopterogorgia elisabethae.

5. The composition of claim 1, wherein the isolated pseudopterosin-producing alga is of the genus Symbiodinium.

6. The composition of claim 5, wherein the isolated pseudopterosin-producing alga is a clade B genus Symbiodinium.

7. The composition of claim 6, wherein the isolated pseudopterosin-producing alga is Symbiodinium microadricum.

8. The composition of claim 1, wherein the isolated pseudopterosin-producing alga is cultured in ASP-8A culture medium.

9. The composition of claim 8, wherein the isolated pseudopterosin-producing alga cells in culture medium, are subjected to illumination using a 14-10 hour light-dark cycle.

10. The composition of claim 9, wherein the light intensity of the illumination is about 30 up to 150 μmol photons m⁻²·s⁻¹.

11. The composition of claim 10, wherein the light intensity of the illumination is about 30 μmol photons m⁻²·s⁻¹.

12. The composition of claim 10, wherein the light intensity of the illumination is about 50 μmol photons m⁻²·s⁻¹.

13. The composition of claim 10, wherein the light intensity of the illumination is about 75 μmol photons m⁻²·s⁻¹.

14. The composition of claim 10, wherein the light intensity of the illumination is about 100 μmol photons m⁻²·s⁻¹.

15. The composition of claim 10, wherein the light intensity of the illumination is 150 μmol photons m⁻²·s⁻¹.

16. The composition of claim 8, wherein the isolated pseudopterosin producing alga in culture medium are maintained at a temperature from about 20° C. to about 30° C.

17. The composition of claim 8, wherein the isolated pseudopterosin producing alga in culture medium are maintained at a temperature between 23° C. to 29° C.

18. The composition of claim 1, wherein the isolated pseudopterosin producing alga in culture medium are free of contaminating microorganisms.

19. The composition of claim 18, wherein isolated DNA from the isolated pseudopterosin producing alga in culture medium is amplified by primers identified by any one of SEQ ID NO's: 1-4.

20. The composition of claim 1, wherein the isolated pseudopterosin producing alga in culture medium produce about 100% more pseudopterosin as compared to an initial isolated pseudopterosin-producing alga.

21. The composition of claim 1, wherein the isolated pseudopterosin producing alga in culture medium produce about 200% more pseudopterosin as compared to an initial isolated pseudopterosin-producing alga.

22. The composition of claim 1, wherein the isolated pseudopterosin producing alga in culture medium produce about 400% more pseudopterosin as compared to an initial isolated pseudopterosin-producing alga.

23. The composition of claim 1, wherein the isolated pseudopterosin producing alga in culture medium produce about 800% more pseudopterosin as compared to an initial isolated pseudopterosin-producing alga.

25. The composition of claim 1, wherein the isolated pseudopterosin producing alga in culture medium produce about 1000% more pseudopterosin as compared to an initial isolated pseudopterosin-producing alga.

26. The composition of claim 20, wherein pseudopterosin levels are measured by HPLC.

27. The composition of claim 1, wherein pseudopterosin-producing alga produce pseudopterosins A-L.

28. The composition of claim 1, wherein cultured pseudopterosin-producing alga produce pseudopterosins A-D.

29. A culture comprising a pseudopterosin-producing alga.

30. The culture of claim 29, wherein the alga is of the genus Symbiodinium.

31. The culture of claim 29, wherein the alga is Symbiodinium microadricum.

32. The culture of claim 29, wherein the alga is obtained from a host.

33. The culture of claim 29, wherein the alga is isolated from a host of the genus Pseudopterogorgia.

34. The culture of claim 29, wherein the alga is isolated from the host, Pseudopterogorgia elisabethae.

35. The culture of claim 31, wherein the Symbiodinium is of clade B.

36. The culture of claim 29, wherein the cell culture medium is ASP-8A.

37. The culture of claim 29, wherein the culture has been subjected to illumination using a 14-10 light-dark cycle.

38. The culture of claim 29, wherein the light intensity of the illumination is 30-150 μmol photons·m⁻²·s⁻¹.

39. The culture of claim 29, wherein the culture is maintained at a temperature between 23-29° C.

40. A method for producing pseudopterosins from isolated pseudopterosin-producing alga cells in a culture medium.

41. The method of claim 40, wherein the pseudopterosin-producing alga is isolated from a host organism.

42. The method of claim 41, wherein the pseudopterosin-producing alga is isolated from a host of the genus Pseudopterogorgia.

43. The method of claim 42, wherein the pseudopterosin-producing alga is isolated from the host, Pseudopterogorgia elisabethae.

44. The method of claim 40, wherein the isolated pseudopterosin-producing alga is of the genus Symbiodinium.

45. The method of claim 44, wherein the isolated pseudopterosin-producing alga is a clade B genus Symbiodinium.

46. The method of claim 45, wherein the isolated pseudopterosin-producing alga is Symbiodinium microadricum.

47. The method of claim 40, wherein the isolated pseudopterosin-producing alga is cultured in ASP-8A culture medium.

48. The method of claim 47, wherein cultured pseudopterosin-producing alga cells are subjected to illumination using a 14-10 hour light-dark cycle.

49. The method of claim 48, wherein the light intensity of the illumination is about 30 up to 150 μmol photons·m⁻²·s⁻¹.

50. The method of claim 49, wherein the light intensity of the illumination is about 30 μmol photons·m⁻²·s⁻¹.

51. The method of claim 49, wherein the light intensity of the illumination is about 50 μmol photons·m⁻²·s⁻¹.

52. The method of claim 49, wherein the light intensity of the illumination is about 75 μmol photons·m⁻²·s⁻¹.

53. The method of claim 49, wherein the light intensity of the illumination is about 100 μmol photons·m⁻²·s⁻¹.

54. The method of claim 49, wherein the light intensity of the illumination is 150 μmol photons·m⁻²·s⁻¹.

55. The method of claim 47, wherein the cultured pseudopterosin producing alga are maintained at a temperature from about 20° C. to about 30° C.

56. The method of claim 47, wherein the cultured pseudopterosin producing alga are maintained at a temperature between 23° C. to 29° C.

57. The method of claim 40, wherein cultured pseudopterosin producing alga are free of contaminating microorganisms.

58. The method of claim 57, wherein isolated DNA from cultured pseudopterosin producing alga is amplified by primers identified by any one of SEQ ID NO's: 1-42.

59. The method of claim 40, wherein cultured pseudopterosin-producing alga produce about 100% more pseudopterosin as compared to an initial isolated pseudopterosin-producing alga.

60. The method of claim 40, wherein cultured pseudopterosin-producing alga produce about 200% more pseudopterosin as compared to an initial isolated pseudopterosin-producing alga.

61. The method of claim 40, wherein cultured pseudopterosin-producing alga produce about 400% more pseudopterosin as compared to an initial isolated pseudopterosin-producing alga.

62. The method of claim 40, wherein cultured pseudopterosin-producing alga produce about 800% more pseudopterosin as compared to an initial isolated pseudopterosin-producing alga.

63. The method of claim 40, wherein cultured pseudopterosin-producing alga produce about 1000% more pseudopterosin as compared to an initial isolated pseudopterosin-producing alga.

64. The method of claim 40, wherein pseudopterosin levels are measured by HPLC.

65. The method of claim 40, wherein pseudopterosin-producing alga produce pseudopterosins A-L.

66. The method of claim 40, wherein cultured pseudopterosin-producing alga produce pseudopterosins A-D.


SEQUENCE LISTING (4 sequences)

SEQ ID NO: 1 - DNA, 29 bp, Zooxanthallae
  PCR_primer (1)..(29)
ggttgatcctgccagtagtcatatgcttg

SEQ ID NO: 2 - DNA, 30 bp, Zooxanthallae
agcactgcgtcagtccgaataaatcaccgg

SEQ ID NO: 3 - DNA, 20 bp, Algae
  PCR_primer (1)..(20)
gcatcgatgaagaacgcagc

SEQ ID NO: 4 - DNA, 18 bp, Algae
  PCR_primer (1)..(18)
gctgcgttcttcagcgat